(19) United States
(12) Patent Application Publication
(10) Pub. No.: US 2009/0047314 A1
(43) Pub. Date: Feb. 19, 2009

(54) MICROENCAPSULATION SYSTEMS AND APPLICATIONS OF SAME

(75) Inventors: Amelie BOCHOT, Paris (FR); Huguette Alphandary, Sceaux (FR); Dominique Duchene, Paris (FR); Elias Fattal, Paris (FR)

Correspondence Address: NIXON & VANDERHYE, PC, 901 NORTH GLEBE ROAD, 11TH FLOOR, ARLINGTON, VA, 22203, US

(73) Assignees: Centre National de la Recherche Scientifique (C.N.R.S.), Paris (FR); Universite Paris XI, Orsay Cedex (FR)

(21) Appl. No.: 12/195,480

(22) Filed: Aug. 21, 2008

Related U.S. Application Data
(63) Continuation of application No. 10/542,703, filed on Mar. 29, 2006, now abandoned.

(30) Foreign Application Priority Data
Jan. 20, 2003 (FR) 03 005 78

(51) Int. Cl. A61K 8/02; A61K 9/14; A61K 35/12; A61K 36/00; A61K 36/736; A61K 36/899; A61P 17/00

(52) U.S. Cl. 424/401; 424/493; 424/522; 424/725; 424/735; 424/750

(57) ABSTRACT

The invention relates to microencapsulation systems which are characterised in that they are developed from oily substances or sugars and in that they form an essentially organised assembly corresponding to stacks of crystalline structures. The inventive systems can be used for the microencapsulation of one or more substances which are of interest, such as therapeutically-active substances, or which can be used in cosmetics.

This is a continuation of application Ser. No. 10/542,703, filed Mar. 29, 2006 (pending), which is a U.S. national phase of International Application No. PCT/FR2004/000119, filed 20 Jan. 2004, which designated the U.S. and claims benefit of FR 03/00578, filed 20 Jan. 2003, the entire contents of each of which is hereby incorporated by reference in this application.

The invention relates to systems for encapsulating substances of interest, and to the applications thereof.

Microencapsulation includes all technologies that make it possible to prepare individualized microbeads consisting of a coating material containing an active material. The microbeads, also called microparticles, have a size of between 1 μm and several mm and typically contain between 5 and 90% (by weight) of active material. The active materials are of very varied origin: pharmaceutical active principles, cosmetic active agents, food additives, plant protection products, fragranced essences, microorganisms, cells or else catalysts of chemical reactions. The coating materials are polymers of natural or synthetic origin, which may be hydrophobic or hydrophilic, or lipids.

Microbeads prepared from hydrophobic polymer materials are generally prepared by phase separation techniques (coacervation or extraction-solvent evaporation) or by polymerization or polycondensation. Phase separation techniques generally use organic solvents that have a certain number of drawbacks: elimination into the atmosphere, a persistence in galenic systems, denaturation of certain microencapsulated molecules. Polymerization or polycondensation methods, while they have the advantage of not using a solvent, have the drawback of using very reactive materials capable of reacting with the substances encapsulated in the microbeads. Finally, most of the materials that make up these starting materials are synthetic substances, the harmful effects of which on the environment or the organism are still not known.

Microbeads formed from hydrophilic polymer materials are generally prepared by gelling or coacervation techniques. This technique, which makes it possible to encapsulate molecules in liquid or solid form, is based on the desolvatation of macromolecules, resulting in phase separation within a solution. In general, with hydrophilic polymers, a complex coacervation is carried out, in which the desolvatation takes place on two polymers. It can, for example, be carried out by adjusting the pH of the solution containing the polymers such that the positive charges of the first polymer balance out the negative charges of the second, forming a precipitation and a coating of the materials to be encapsulated. The gelled membrane is then crosslinked with glutaraldehyde. This technique is applicable especially to lipophilic materials (plant or mineral oils, essential oils). The microbeads can be prepared by ionic gel formation. In this case, a solution of sodium alginate or pectinate is injected (by prilling) into a solution of calcium chloride. Upon contact with this solution, the drops gel, forming microbeads.

As regards the use of lipid materials, the microencapsulation is carried out by thermal gel formation. This method, called “hot melt”, is based on the melting of the coating material. The active material to be encapsulated is dissolved or dispersed in this molten material. The combination is emulsified in a dispersant phase, the temperature of which is maintained above the melting temperature of the coating. Solidification of the dispersed globules is obtained by abruptly cooling the medium.

Alongside this type of particulate microencapsulation, soft phases (micelles, liposomes, spherulites, microemulsions, emulsions, etc) and molecular encapsulation (cyclodextrins) are distinguished.

The inventors' studies in this field have shown that it is possible to form novel systems that can be used to trap substances of interest by simple orbital agitation, at ambient temperature or close to ambient temperature, using compounds capable of interacting with oily substances.

The aim of the invention is therefore to provide novel microencapsulation systems that are highly stable with respect to storage, having in particular a high sensitivity to shear, which makes it possible to readily release their contents.

A subject of the invention is also the applications of these systems, in particular in therapeutics, in cosmetics and in the food sector.

The microencapsulation systems of the invention are characterized in that they are developed from oily substances and from sugars, and form an essentially organized assembly.

This organization corresponds more particularly to stacks of crystalline structures. Systems of this type exhibit, for example, an organization in the form of hexagonal- or pseudohexagonal-type crystalline structures.

The term “sugar”, as used in the description and the claims, denotes polysaccharides and/or oligosaccharides, and/or starches, and/or derivatives thereof.

In a preferred embodiment of the invention, said sugars are oligosaccharides, and in particular cyclodextrins and derivatives thereof.

α-Cyclodextrin is particularly advantageous given its ability to form inclusion complexes with oily substances.

In other embodiments of the invention, said sugars are polysaccharides, such as starch.

The various sugars and oily substances above correspond to natural or synthetic molecules.

The oily substances that go toward making up the composition of the systems of the invention are liquids or semi-solids and are capable of forming the oily phase of an emulsion. Mention will more especially be made of oils or constituents thereof. These are in particular fatty acids, monoglycerides, diglycerides or triglycerides.

Suitable oils comprise plant oils, such as soya oil, wheatgerm oil, avocado oil or sweet almond oil, or animal oils, such as onager oil, synthetic oils or mineral oils, such as paraffin oil.

In the systems defined above, the oily substances may be in the dispersed state and/or in the form of inclusion complexes, for example with cyclodextrins, and in particular α-cyclodextrin.

Substances of interest can be trapped in said oily substances.

The invention is therefore directed toward the systems containing, in addition, one or more substances of interest chosen from substances that do not affect the organization of the assembly and its stability.

These substances of interest are water-soluble substances or liposoluble substances.

The invention advantageously makes it possible to formulate fragile molecules, that are sensitive to oxidation or to light, or that may be denatured by conventional encapsulation methods, which make use of organic solvents and/or of surfactants, the complete extraction of which is difficult, or even impossible, at a high temperature, or else at shears that are too great.

According to one embodiment of the invention, the systems of the invention are provided in particular in the form of solid beads with a dense structure. Such beads generally have a particle size of one micron to several centimeters, in particular of 0.1 to 8 mm, or else of 0.1 to 5 mm, in particular of 0.5 to 3 mm.

In another embodiment of the invention, the systems are provided in the form of compact or fluid phases.

These various systems can also be dried, lyophilized, or suspended in an aqueous or nonaqueous medium, that is liquid or gelled.

In the form of dried beads, which may or may not be lyophilized, the systems of the invention can be introduced into gelatin capsules.

The invention is also directed toward a method for preparing the systems defined above.

This method is characterized in that it comprises the steps:

consisting of addition of oily substances to an aqueous solution or suspension of a sugar capable of interacting with said oily substances by forming the systems of the invention;

consisting of moderate agitation of the medium, at a temperature of 15 to 40° C., preferably of 18 to 37° C., more particularly of 20 to 30° C., especially of 20 to 25° C., and consisting of recovery of the systems formed.

The agitation is carried out under conditions of speed and of duration that make it possible to obtain solid beads of dense structure, the latter being recovered, washed and optionally dried or lyophilized. As a variant, the agitation is stopped before the formation of these beads, and the intermediate phases are recovered, more especially the compact phase defined above.

To improve the solubility of the molecules of interest, the use of a co-solvent can be envisioned.

Advantageously, this method resorts to neither the use of organic solvents, nor to a heating step, nor to a large consumption of energy, which constitutes an advance of great interest in the encapsulation field.

It will be noted that this method for producing the beads does not require any special equipment for the production, such as specific turbines, homogenizers or hoods. The agitation required to form the beads consumes only a very small amount of energy. The method of production does not involve organic solvents or surfactants, which represents an advantage in terms of safety. The materials employed for forming the beads and the intermediate phases are nontoxic and biodegradable (oily substances, sugars). It is possible to form beads with these sugars, especially polysaccharides and oligosaccharides, and in particular cyclodextrins without crosslinking. The materials used are readily available on the market at a moderate cost.

The invention thus provides highly simple and inexpensive means for producing novel systems that can be used in many sectors of the industry.

The invention is directed in particular toward the application thereof in therapeutics, where they make it possible in particular to encapsulate active principles of medicinal products, and constitute novel galenic forms or any intermediate form that can be used in the preparation of other administration forms (gelatin capsules, granules, compact powders, etc) for oral administration. The active principles encapsulated according to the invention can also be administered cutaneously or onto the mucous membranes.

The invention is also directed in particular toward the application thereof in cosmetics, for encapsulating substances that are active in cosmetology and/or pigments and/or dyes and natural or synthetic products that go toward making up the composition of perfumes, aromas, fragrances. The use of these systems thus makes it possible to prepare novel formulations that can be used, for example, as make-up products. Forms and presentations such as compacts, sticks of beads, fluid gels of beads, bath pearls, or the like, can thus be developed.

Another application of interest concerns the food sector. Novel formulations of dietetic products, foods or medicinal foods can be prepared.

It will be noted that, in these applications, the systems have the advantage of masking unpleasant odors or tastes.

Mention may also be made of the application of the systems of the invention in the agronomics industries, for example for encapsulating pesticides, or paints containing mineral or organic pigments using various types of binders (water-based, oil-based, etc) in liquid or paste form, paint in the dry state (crayons, pastels, particulate powder, etc), oily coatings.

Other characteristics and advantages of the invention will be given in the following examples, which relate to embodiments of the invention involving, by way of illustration, α-cyclodextrin as oligosaccharide, and plant or animal oils as oily substances.

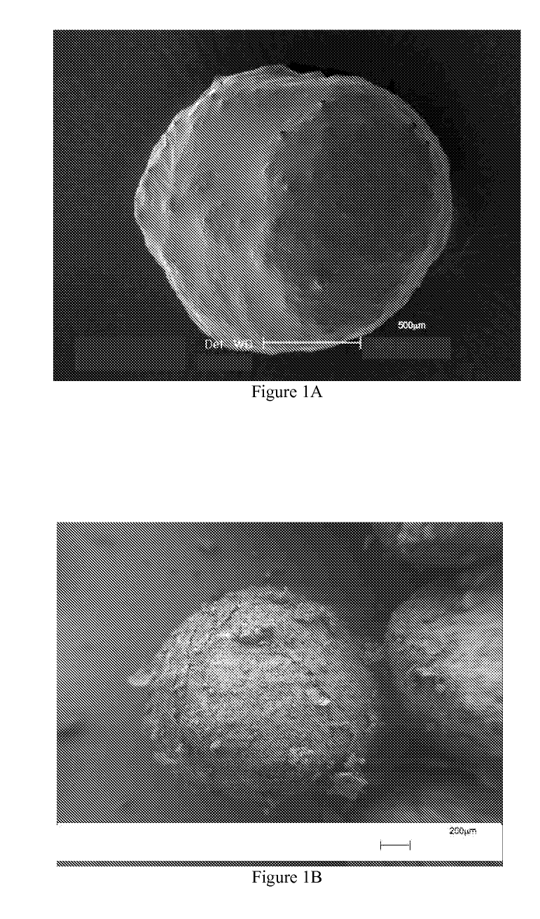

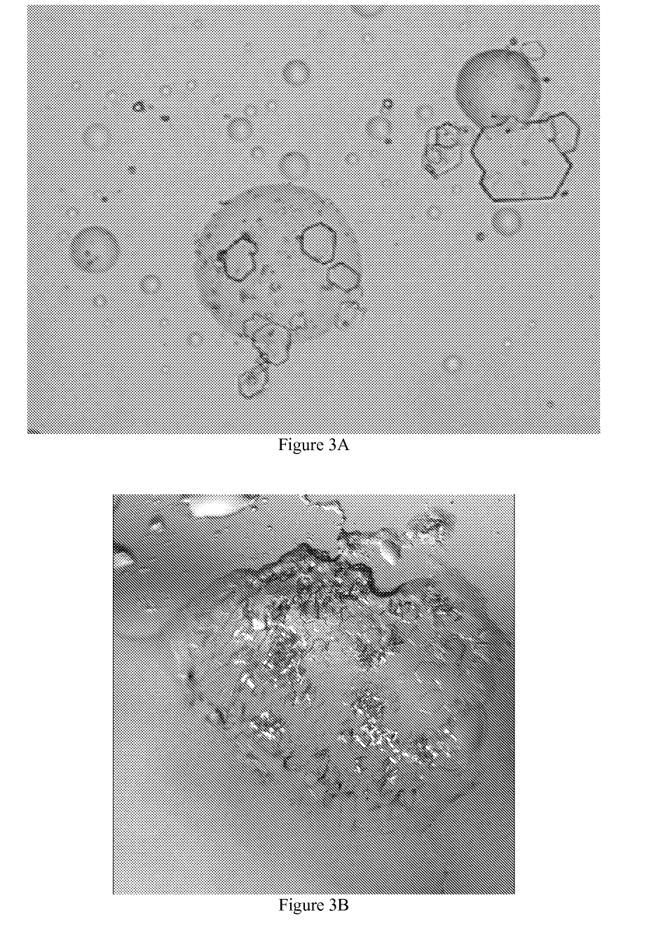

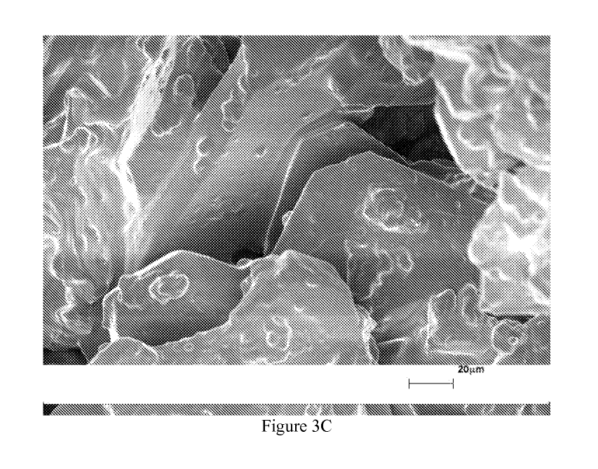

In these examples, reference will be made to FIGS. 1 to 5, which represent, respectively,

FIGS. 1a to 1c: photographs from scanning electron microscopy on whole beads before lyophilization (FIG. 1a), and that had been lyophilized (FIG. 1b): (Mag×30) and on their surface (Mag×625) (FIG. 1c);

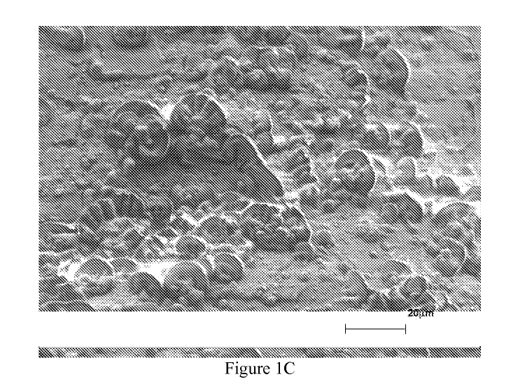

FIGS. 2a and 2b: photographs from transmission electron microscopy carried out on a cryofracture of beads (Mag×30 000) FIG. 2a); the area in zoom (Mag×78 000) (FIG. 2b);

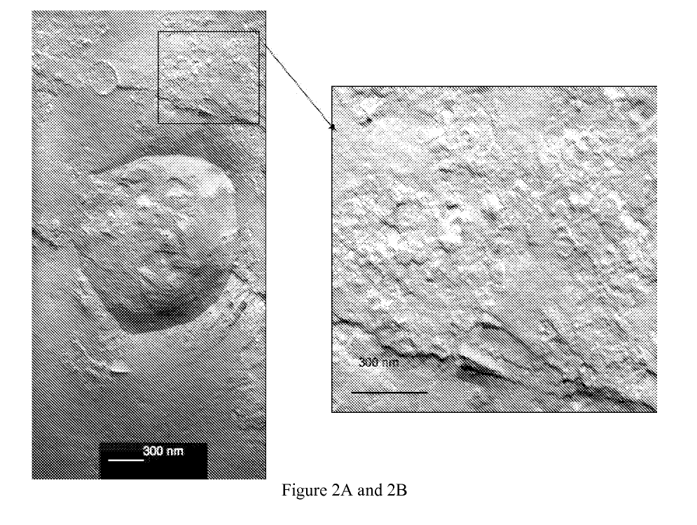

FIGS. 3a to 3c: a photograph of crystals observed by optical microscopy (Mag×650) (FIG. 3a); a photograph from confocal optical microscopy on semi-thin sections of beads labeled with Nile Red, embedded in resin, transmission image (Mag×64) (FIG. 3b), and a photograph from scanning electron microscopy on crystals after extraction with isopentane (Mag×4000) (FIG. 3c).

### EXAMPLE 1

### Formation Of Beads From α-Cyclodextrin And Plant Oils

In a first step, cyclodextrin (α-CD) (3 to 6% m/m) possibly solubilized in an aqueous phase representing 67 to 82% of the total mass is introduced into a flask. An oily phase formed from soya oil (15 to 30% m/m) is added at the surface of the water. The pH of the aqueous phase can be adjusted from pH 2 to 9.3. The molecule to be encapsulated can be added to one of the two phases: a water-soluble molecule can be added to the aqueous phase and a liposoluble molecule can be added to the oily phase. The flask is then stoppered, and then subjected to agitation (Rotatest, Bioblock Scientific) at a speed of 200 rpm, in a thermostatted water bath (28° C.). After a period of approximately 0.5 to 30 days, but more generally of 2 to 3 days, white-colored, more or less spherical beads form. Several intermediate states (fluid and then compact states) precede the formation of the beads. The kinetics of bead formation, under the conditions tested, is slower at acidic pHs. At pHs of 9.5 to 10.3, the phases remain compact.

By carrying out the procedure with concentrations of soya oil of 12-24% m/m, of osmosed water of 70-82% m/m and of α-CD of 3.3-6% m/m, beads of 0.5 to 3 mm, and a clear suspension medium exhibiting few, if any, oily globules, are obtained in 0.5 to 5 days.

For the tests hereinafter, a ternary mixture of 2.88 ml of soya oil, 10 ml of osmosed water of pH 5.5-6, and 0.813 g of α-cyclodextrin was used.

The beads obtained are stable (for at least 3 years) and in suspension in a dispersing phase whose turbidity varies. In fact, the beads prepared under the conditions above exhibit a homogeneous size distribution and are in a clear dispersant phase. The beads that exhibit a more heterogeneous size distribution are in a whitish phase.

The beads in suspension in water, dried or lyophilized, can be dispersed in hydrogels, for example made of Carbomer, of cellulose or of poloxamer 407.

It will be noted that these treatments, in particular the drying and the lyophilization thereof, do not impair their characteristics, which is advantageous in terms of their conservation.

The beads are capable of undergoing other operations such as filtration at normal pressure, low-speed centrifugation, drying an oven (the beads then become transparent).

FIGS. 1a-1c show the photographs from scanning electron microscopy of the surface of a bead according to the invention before lyophilization (FIG. 1a), of a lyophilized bead (FIG. 1b), (Mag×30), and a view of the surface (Mag×625) (FIG. 1c). This examination shows a surface with rough patches, whether or not the beads are lyophilized.

The internal structure of the beads was also studied. To this effect, the beads in suspension in water were subjected to cryofracture and the replicas were observed by transmission electron microscopy. As shown in FIGS. 2a and 2b, the beads have a matrix structure, i.e. dense structure, exhibiting globular structures and regular, angular elements of approximately 30 nm.

The beads consist of lipophilic (oil) and hydrophilic (cyclodextrin) compartments. The images obtained by confocal microscopy show calceine (hydrophilic fluorescent label) distribution at the surface of the beads and sporadic distribution of Nile Red (fluorescent label for lipids) at the surface of and inside said beads. Microscopic analysis of the bead suspension media does not demonstrate any substantial presence of oil droplets, showing that the oil is indeed trapped in the system.

he presence of many pseudohexagonal crystals, of heterogeneous size ranging from 10 nm to a few microns, within the beads was shown by optical microscopy (FIG. 3a), confocal microscopy (FIG. 3b) and scanning electron microscopy (FIG. 3c). It was possible to isolate these crystals by treatment with isopentane and this could be demonstrated by scanning electron microscopy, transmission electron microscopy (cryofracture, negative staining, ultrathin sections, electron diffraction) and by small-angle and wide-angle X-ray diffraction.

### Stability Of The Beads In Biological Media

In the perspective of oral administration of the beads containing active principles, tests of stability of the lyophilized and nonlyophilized beads were carried out in media simulating digestive liquids, subjected to agitation at 37° C. (stomach medium, pH 1.2; intestines, pH 6.8: media described by the American Pharmacopoeia USP XXIII).

The beads are stable for approximately 5 h 30 min in the stomach medium and approximately 4 h 30 min in the intestinal medium. Beyond these times, a decrease in the number of beads and in their size is observed. Virtually similar results were recorded with the lyophilized beads and nonlyophilized beads.

### EXAMPLE 2

### Encapsulation Of Molecules In The Beads

The procedure is carried out as described in Example 1, but using, as molecules of interest, molecules that are active in therapeutics or that can be used in cosmetics, such as pigments or dyes, vitamin E acetate, benzophenone or isotretinoin.

The table below gives the diameter of the beads obtained and the formation time

| | | Bead | Formation |
|---|---|---|---|
| Molecules | Concentration | diameter | time |
| Pharmacy | | | |
| 5-Methoxypsoralene | 0.52 mg/ml oil | 2 mm | 2 days |
| Cosmetics | | | |
| Vitamin E acetate | 23.4 mg/ml oil | 2 mm | 3 days |
| Vitamin E acetate | 46.9 mg/ml oil | 2 mm | 4 days |
| Vitamin E | 23.4 mg/ml oil | 2 mm | 7 days |
| benzophenone | 1.9 mg/ml oil | 2 mm | 3 days |
| Fluorescent label | | | |
| Calceine | 0.3 mg/ml water | 2 mm | 7 days |
| Nile Red | | 2 mm | 3 days |
| Liopsoluble dyes | | | |
| Chromium oxide | | | 3 days |
| (green) | | | |
| Methyl yellow | 5.1 mg/ml oil | | 4 days |
| Cobalt salt (blue) | | | 3 days |
| Mica, titanium | | | 5 days |
| dioxide, iron oxide | | | |
| Water-soluble dyes | | | |
| Methylene blue | | | 4 days |
| Various | | | |
| Cacao | 2.7 mg/ml oil | 2.5 mm | 7 days |

It is noted that the presence of the lipophilic or hydrophilic molecules tested does not modify the characteristics of the beads, either with respect to their size or with respect to their formation time. In addition, it was shown that 30% of vitamin E acetate is encapsulated (determination by HPLC).

Beads containing fragrances, for example Femme® by Rochas, were also prepared.

### EXAMPLE 3

### Formation Of The Beads In The Presence Of Co-Solvent

The procedure was carried out as described in Example 1, but adding a co-solvent to the oil or to the water.

| Co-solvent | Bead diameter | Formation time |
|---|---|---|
| Ethanol (200 microl | 1 mm | 9 days |
| in 2.68 ml of oil) | | |
| Miglyol 810 (200 | 1 mm | 3 days |
| microl in 2.68 ml | | |
| of oil) | | |
| 5% glycerol in | 3 mm | 3 days |
| osmosed water | | |
| 10% glycerol in | 2 mm | 3 days |
| osmosed water | | |
| 15% glycerol in | 1.5 mm | 3 days |
| osmosed water | | |

### EXAMPLE 4

### Formation Of The Beads After Pre-Emulsification Of The Aqueous Phase With The Oily Phase

The procedure was carried out as described in Example 1, but the aqueous phase containing the cyclodextrin (α) was emulsified with the oily phase using a turbine agitator. The mixture obtained was then subjected to the agitation conditions described in Example 1.

1. A microencapsulation system, characterized in that it is developed from oily substances and from sugars, and forms an essentially organized assembly corresponding to stacks of crystalline structures.

2. The system as claimed in claim 1, characterized in that it exhibits an organization in the form of hexagonal- or pseudohexagonal-type crystalline structures.

3. The system as claimed in claim 1, characterized in that the sugars are polysaccharides and/or oligosaccharides, and/or starches, and/or derivatives thereof.

4. The system as claimed in claim 3, characterized in that the oligosaccharides are cyclodextrins.

5. The system as claimed in claim 4, characterized in that the cyclodextrin is α-cyclodextrin.

6. The system as claimed in claim 1, characterized in that the oily substances are fatty acids, monoglycerides, diglycerides or triglycerides.

7. The system as claimed in claim 6, characterized in that the oily substances are plant oils, such as soya oil, wheatgerm oil, avocado oil or sweet almond oil, animal oils, such as onager oil, or synthetic oils or mineral oils.

8. The system as claimed in claim 1, characterized in that said oily substances are in the dispersed state and/or in the form of inclusion complexes, for example with cyclodextrins, and in particular α-cyclodextrin.

9. The system as claimed in claim 1, characterized in that it contains, in addition, one or more substances of interest.

10. The system as claimed in claim 9, characterized in that the substances of interest are water-soluble substances or liposoluble substances.

11. The system as claimed in claim 10, characterized in that said substance(s) is (are) therapeutically active, in particular at low dose.

12. The system as claimed in claim 10, characterized in that said substance(s) can be used in the cosmetics field.

13. The system as claimed in claim 1, characterized in that it is provided in the form of solid beads with a dense structure.

14. The system as claimed in claim 13, characterized by a particle size of one micron to several centimeters, in particular of 0.1 to 8 mm, or else of 0.1 to 5 mm, in particular of 0.5 to 3 mm.

15. The system as claimed in claim 1, characterized in that it is provided in the form of compact phases.

16. The system as claimed in claim 1, characterized in that it is provided in the form of beads in suspension, or of dried or lyophilized beads, which may or may not be redispersed in an aqueous or nonaqueous liquid or in a gel.

17. A method for preparing the system as claimed in claim 1, characterized in that it comprises the steps:
consisting of addition of oily substances to an aqueous solution or suspension of sugar capable of interacting with said oily substances by forming essentially organized systems corresponding to stacks of crystalline structures, in particular hexagonal- or pseudohexagonal-type structures;
consisting of moderate agitation of the medium, at a temperature of 15 to 40° C., preferably of 18 to 37° C., more particularly of 20 to 30° C., especially of 20 to 25° C., and consisting of recovery of the systems formed.

18. The method as claimed in claim 17, characterized in that the agitation is carried out under conditions of speed and of duration that make it possible to obtain solid beads with a dense structure, or a compact or fluid phase.
